(19) United States
(12) Patent Application Publication
(10) Pub. No.: US 2009/0176698 A1
(43) Pub. Date: Jul. 9, 2009

(54) Benzimidazolone Derivatives for the Treatment of Urinary Incontinence

(75) Inventors: Wolfgang Baiker, Volxheim (DE); Angelo Ceci, Mittelbiberach (DE)

Correspondence Address: Michael P. Morris;Boehringer Ingelheim USA Corporation, 900 Ridgebury Road, Ridgefield, CT, 06877-0368, US

(73) Assignee: BOEHRINGER INGELHEIM INTERNATIONAL GMBH, Ingelheim (DE)

(21) Appl. No.: 12/279,870

(22) PCT Filed: Feb. 16, 2007
(86) PCT No.: PCT/EP07/51494

§ 371 Date: Oct. 10, 2008

(30) Foreign Application Priority Data
Feb. 20, 2006 (EP) 06003371.9

(51) Int. Cl. A61K 38/12; A61K 31/496; A61K 31/138; A61K 31/216; A61K 31/4725; A61K 31/137; A61K 31/381; A61K 31/5375; A61P 13/02

(52) U.S. Cl. 514/11; 514/254.06; 514/654; 514/534; 514/305; 514/649; 514/438; 514/239.2

(57) ABSTRACT

The invention relates to compositions comprising benzimidazolone derivatives of formula (I), optionally in form of the free base or in form of the pharmacologically acceptable acid addition salts thereof and methods of treating or preventing urinary incontinence, comprising the administration of a therapeutically effective amount of compound of formula (I), wherein R₁, R₂, R₃, and R₄ denote hydrogen or hydroxy with the proviso that R₁, R₂, R₃, and R₄ cannot simultaneously represent hydrogen, optionally in form of the free base or in form of the pharmacologically acceptable acid addition salts thereof.

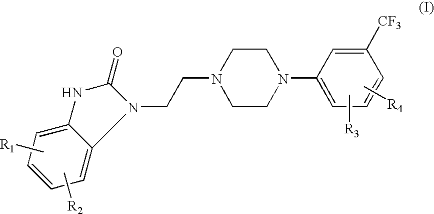

The invention relates to compositions comprising benzimidazolone derivatives of formula (I), optionally in form of the free base or in form of the pharmacologically acceptable acid addition salts thereof and methods of treating or preventing urinary incontinence, comprising the administration of a therapeutically effective amount of a compound of formula (I).

### DESCRIPTION OF THE INVENTION

The compounds of formula (I), their free bases and their acid addition salts are disclosed in WO 01/21593 A1 and have the following chemical structure:

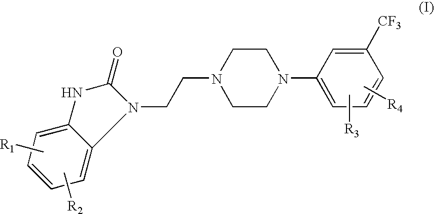

wherein R₁, R₂, R₃, and R₄ denote hydrogen or hydroxy with the proviso that R₁, R₂, R₃, and R₄ cannot simultaneously represent hydrogen.

Preferred compounds according to the invention are those of general formula (I) wherein two or three of the four radicals R1, R2, R3, and R4 denote hydrogen. Also preferred are compounds of general formula (I) wherein one of the radicals R1, R2, R3, and R4 denotes hydroxy, whilst the other radicals represent hydrogen.

Above mentioned compounds show affinity for the 5-HT1A and 5-HT2-receptor. They may be of value in the treatment of those diseases where an altered functioning of neurosignal transmission is present. Examples of these CNS disorders include depression, schizophrenia, Parkinson, anxiety, sleep disturbances, sexual and mental disorders and age associated memory impairment (WO 01/21593 A1).

In one embodiment the present invention relates to methods of treating or preventing urinary incontinence, comprising the administration of a therapeutically effective amount of one or more, preferably one compound of formula (I) 1, optionally in form of the free base or in form of the pharmacologically acceptable acid addition salts thereof.

Urinary incontinence may derive from functional bladder problems, a heterogeneous group of disorders which differ in their aetiology, diagnosis and therapy. In the standardising recommendations of the International Continence Society (ICS) urinary incontinence is defined as involuntary loss of urine which is objectively detectable and constitutes a social and hygiene problem. Generally, urinary incontinence only occurs when there is an unintentional increase of pressure in the bladder during the storage phase. This can happen as a result of unrestricted contractions of the detrusor muscle (urge incontinence) or failure of the urethral closure mechanism (stress incontinence).

Urge incontinence is one of the symptoms which is categorised under the syndrome of Overactive Bladder (OAB). According to the ICS definition, OAB is characterised by an irresistible imperative need to urinate, which may or may not be associated with urge incontinence, usually with increased frequency of micturition and nocturnal urination. Pathophysiologically, this complaint may be based on involuntary detrusor contractions during the filling phase, the cause of which may be neurogenic or non-neurogenic (idiopathic) by nature. Uresiesthesis and urge incontinence are extremely unpleasant and troublesome to those affected, leading to considerable impairment of their quality of life and psychological, professional, domestic, physical and sexual problems.

Stress incontinence is characterised by the involuntary loss or urine which generally occurs at moments of elevated intraabdominal pressure. This may occur for example when lifting, coughing, sneezing, running while at the same time there is no detrusor activity. Loss of urine takes place as the result of a variable combination of an insufficiency of the sphincter muscles of the bladder and the pelvic floor as well as anatomical defects in the suspensory apparatus. As a result the closure pressure of the urethra is too low and incontinence results. Pure stress incontinence often occurs in women, particularly if they have given birth. In men, this form of urinary incontinence is usually only observed after prostatectomies or other surgical interventions on the small pelvis.

In mixed incontinence patients suffer from symptoms of both stress incontinence and urge incontinence. Again more women are affected than men.

In another embodiment, the present invention relates to methods of treating or preventing urinary incontinence, comprising the administration of a therapeutically effective amount of one or more, preferably one compound of formula (I) 1, wherein 1 is selected from the group consisting of

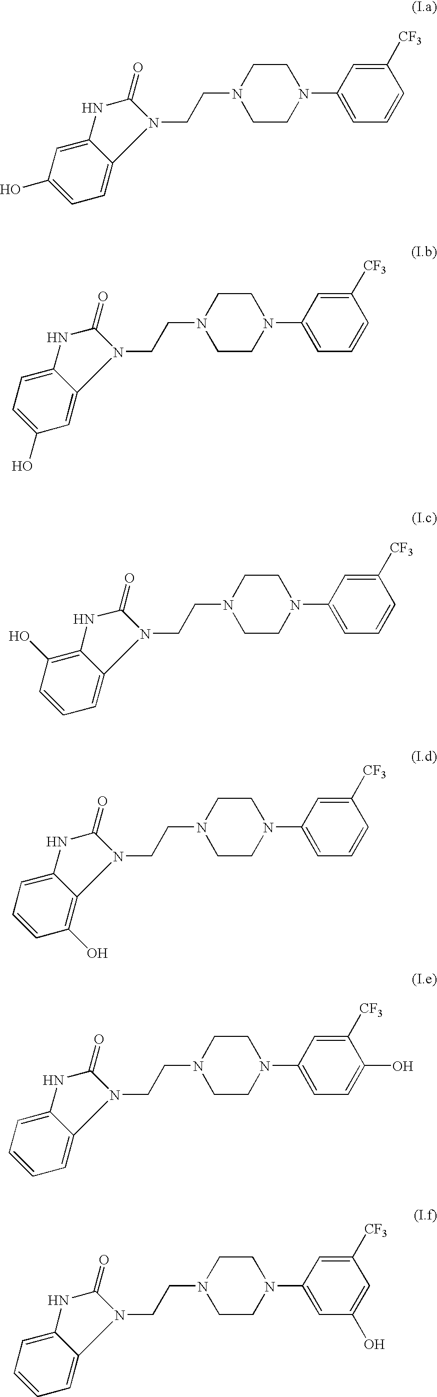

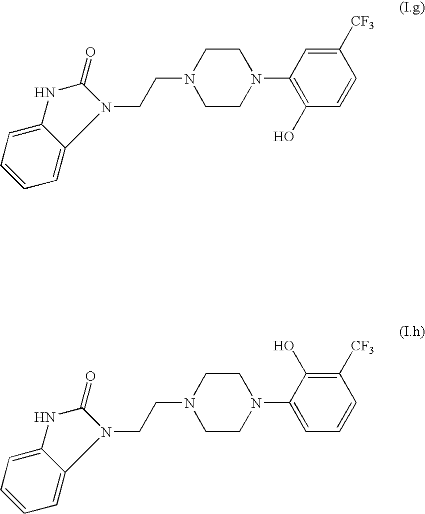

optionally in form of the free base or in form of the pharmacologically acceptable acid addition salts thereof.

In another embodiment the present invention relates to methods of treating or preventing overactive bladder syndrome, comprising the administration of a therapeutically effective amount of one or more, preferably one compound of formula (I) 1, optionally in form of the free base or in form of the pharmacologically acceptable acid addition salts thereof. Preferably the compounds of formula (I) 1 are selected from the group consisting of the compounds (I.a), (I.b), (I.c), (I.d), (I.e), (I.f), (I.g) and (I.h).

In another embodiment the present invention relates to methods of treating or preventing urge incontinence, comprising the administration of a therapeutically effective amount of one or more, preferably one compound of formula (I) 1, optionally in form of the free base or in form of the pharmacologically acceptable acid addition salts thereof. Preferably the compounds of formula (I) 1 are selected from the group consisting of the compounds (I.a), (I.b), (I.c), (I.d), (I.e), (I.f), (I.g) and (I.h).

In another embodiment the present invention relates to methods of treating or preventing stress incontinence, comprising the administration of a therapeutically effective amount of one or more, preferably one compound of formula (I) 1, optionally in form of the free base or in form of the pharmacologically acceptable acid addition salts thereof. Preferably the compounds of formula (I) 1 are selected from the group consisting of the compounds (I.a), (I.b), (I.c), (I.d), (I.e), (I.f), (I.g) and (I.h).

In another embodiment the present invention relates to methods of treating or preventing mixed incontinence, comprising the administration of a therapeutically effective amount of one or more, preferably one compound of formula (I) 1, optionally in form of the free base or in form of the pharmacologically acceptable acid addition salts thereof. Preferably the compounds of formula (I) 1 are selected from the group consisting of the compounds (I.a), (I.b), (I.c), (I.d), (I.e), (I.f), (I.g) and (I.h).

Another embodiment of the present invention relates to the use of the compounds of formula (I) 1, optionally in form of the free base or in form of the pharmacologically acceptable acid addition salts thereof, for the preparation of a medicament for the treatment of any of the aforementioned disorders. Preferably the compounds of formula (I) 1 are selected from the group consisting of the compounds (I.a), (I.b), (I.c), (I.d), (I.e), (I.f), (I.g) and (I.h).

As benzimidazolone derivatives of formula (I) 1 can not only be used as a monotherapy but also in combination with other active ingredients useful for treatment of urinary incontinence, another embodiment of the invention relates to new pharmaceutical compositions comprising a therapeutically effective amount of one or more, preferably one compound of formula (I) 1, optionally in form of the free base or in form of the pharmacologically acceptable acid addition salts thereof as one active ingredient in combination with a therapeutically effective amount one or more, preferably one active ingredient 2 useful for treatment of urinary incontinence. Preferably 1 is selected from the group consisting of the compounds (I.a), (I.b), (I.c), (I.d), (I.e), (I.f), (I.g) and (I.h), optionally in form of the free base or in form of the pharmacologically acceptable acid addition salts thereof.

The compositions according to the invention may contain the compounds of formula (I) 1 and the one or more additional active ingredient 2 in a single formulation or in separate formulations (multiple dosage form). If the compounds of formula (I) 1 and the one or more, preferably one active ingredient 2 are present in separate formulations these separate formulations may be administered simultaneously or sequentially.

In a further embodiment, the present invention is directed to pharmaceutical compositions comprising a therapeutically effective amount of one or more, preferably one compound of formula (I) 1, optionally in form of the free base or in form of the pharmacologically acceptable acid addition salts thereof as one active ingredient in combination with a therapeutically effective amount of one or more, preferably one active ingredient 2 useful for treatment of urinary incontinence, wherein 2 is selected from the group consisting of antimuscarinic agents 2a, vasopressin agonists 2b and Serotonin/Noradrenaline modulators 2c. Preferably 1 is selected from the group consisting of the compounds (I.a), (I.b), (I.c), (I.d), (I.e), (I.f), (I.g) and (I.h).

In a further embodiment, the present invention is directed to pharmaceutical compositions comprising a therapeutically effective amount of one or more, preferably one compound of formula (I) 1, optionally in form of the free base or in form of the pharmacologically acceptable acid addition salts thereof as one active ingredient in combination with a therapeutically effective amount of one or more, preferably one antimuscarinic agent 2a, optionally in form of the pharmaceutically acceptable salts, in form of the hydrates and/or solvates and optionally in the form of the individual optical isomers, mixtures of the individual enantiomers or racemates thereof and optionally in combination with a pharmaceutical acceptable excipient. Preferred antimuscarinic agents 2a include Tolterodine, Oxybutynin, Solifenacin and Trospium. Preferably 1 is selected from the group consisting of the compounds (I.a), (I.b), (I.c), (I.d), (I.e), (I.f), (I.g) and (I.h).

In a further embodiment, the present invention is directed to pharmaceutical compositions comprising a therapeutically effective amount of one or more, preferably one compound of formula (I) 1, optionally in form of the free base or in form of the pharmacologically acceptable acid addition salts thereof as one active ingredient in combination with a therapeutically effective amount of one or more, preferably one vasopressin agonist 2b, optionally in form of the pharmaceutically acceptable salts, in form of the hydrates and/or solvates and optionally in the form of the individual optical isomers, mixtures of the individual enantiomers or racemates thereof and optionally in combination with a pharmaceutical acceptable excipient. A preferred vasopressin agonist 2b is Desmopressin. Preferably 1 is selected from the group consisting of the compounds (I.a), (I.b), (I.c), (I.d), (I.e), (I.f), (I.g) and (I.h).

In a further embodiment, the present invention is directed to pharmaceutical compositions comprising a therapeutically effective amount of one or more, preferably one compound of formula (I) 1, optionally in form of the free base or in form of the pharmacologically acceptable acid addition salts thereof as one active ingredient in combination with a therapeutically effective amount of one or more, preferably one Serotonin/Noradrenaline modulator 2c, optionally in form of the pharmaceutically acceptable salts, in form of the hydrates and/or solvates and optionally in the form of the individual optical isomers, mixtures of the individual enantiomers or racemates thereof and optionally in combination with a pharmaceutical acceptable excipient. Preferred Serotonin/Noradrenaline modulators 2c include Venlafaxine, Duloxetine, Reboxetine and Cizoliritine. Preferably 1 is selected from the group consisting of the compounds (I.a), (I.b), (I.c), (I.d), (I.e), (I.f), (I.g) and (I.h).

The compounds of formula (I) 1 and the compounds (I.a), (I.b), (I.c), (I.d), (I.e), (I.f), (I.g) and (I.h) can be used either as free base or in form of its pharmaceutically acceptable acid addition salts. The term “acceptable acid addition salts includes both organic and inorganic acids such as maleic, citric, tartaric, methanesulphonic, acetic, benzoic, succinic, gluconic, isethionic, glycinic, lactic, malic, mucoic, glutamic, sulphamic and ascorbic acid; inorganic acids include hydrochloric, hydrobromic, nitric, sulfuric or phosphoric acid. Mixtures of the above mentioned acid addition salts may also be used.

The active ingredients 2 which are suitable to be combined with the compound of formula (I) 1 within the teaching of the instant invention and which are mentioned hereinbefore may also be capable of forming acid addition salts with pharmaceutically acceptable acids. Representative salts include the following: Acetate, Benzenesulfonate, Benzoate, Bicarbonate, Bisulfate, Bitartrate, Borate, Bromide, Camsylate, Carbonate, Chloride, Clavulanate, Citrate, Dihydrochloride, Edetate, Edisylate, Estolate, Esylate, Fumarate, Gluceptate, Gluconate, Glutamate, Glycollylarsanilate, Hexylresorcinate, Hydrabamine, Hydrobromide, Hydrochloride, Hydroxynaphthoate, Iodide, Isothionate, Lactate, Lactobionate, Laurate, Malate, Maleate, Mandelate, Mesylate, Methylbromide, Methylnitrate, Methylsulfate, Mucate, Napsylate, Nitrate, N-methylglucamine ammonium salt, Oleate, Oxalate, Pamoate (Embonate), Palmitate, Pantothenate, Phosphate/diphosphate, Polygalacturonate, Salicylate, Stearate, Sulfate, Subacetate, Succinate, Tannate, Tartrate, Teoclate, Tosylate, Triethiodide and Valerate.

Furthermore, where the compounds 2 carry an acidic moiety, suitable pharmaceutically acceptable salts thereof may include alkali metal salts, e.g., sodium or potassium salts; alkaline earth metal salts, e.g., calcium or magnesium salts; and salts formed with suitable organic ligands, e.g., quaternary ammonium salts.

The compounds 2 may have chiral centers and occur as racemates, racemic mixtures and as individual diastereomers, or enantiomers with all isomeric forms being included in the present invention. Therefore, where a compound is chiral, the separate enantiomers, substantially free of the other, are included within the scope of the invention. Further included are all mixtures of the two enantiomers. Also included within the scope of the invention are polymorphs and hydrates of the compounds of the instant invention.

The present invention includes within its scope prodrugs of the compounds 1 and 2. In general, such prodrugs will be functional derivatives of the compounds of this invention which are readily convertible in vivo into the required compound.

The term “therapeutically effective amount” shall mean that amount of a drug or pharmaceutical agent that will elicit the biological or medical response of a tissue, system, animal or human that is being sought by a researcher or clinician.

As used herein, the term “composition” is intended to encompass a product comprising the specified ingredients in the specified amounts, as well as any product which results, directly or indirectly, from combination of the specified ingredients in the specified amounts.

According to the present invention the compounds of formula (I) 1 may be administered as a monotherapy or together with component 2 as a combination therapy. If compound of formula (I) 1 is administered in combination with component 2, 1 and 2 may be administered separately or together in one pharmaceutical composition. In addition, the administration of one element of the combination of the present invention may be prior to, concurrent to, or subsequent to the administration of the other element of the combination.

The compound of formula (I) 1 or the elements of the combination of 1 and 2 may be administered by oral, parenteral (e.g., intramuscular, intraperitoneal, intravenous or subcutaneous injection, or implant), buccal, nasal, vaginal, rectal, sublingual, or topical (e.g. ocular eyedrop) routes of administration and may be formulated, alone or together, in suitable dosage unit formulations containing conventional non-toxic pharmaceutically acceptable carriers, adjuvants and vehicles appropriate for each route of administration.

The pharmaceutical compositions, dosage forms, kit of parts for the administration of 1 or 1 and 2 of this invention may conveniently be presented in dosage unit form and may be prepared by any of the methods well known in the art of pharmacy. All methods include the step of bringing the active ingredient into association with the carrier which is constituted of one or more accessory ingredients. In general, the pharmaceutical compositions, dosage forms, kit of parts are prepared by uniformly and intimately bringing the active ingredients into association with a liquid carrier or a finely divided solid carrier or both, and then, if necessary, shaping the product into the desired dosage form. In the pharmaceutical compositions the active compounds are included in an amount sufficient to produce the desired pharmacologic effect.

The pharmaceutical formulations, compositions, dosage forms or kit of parts containing 1 and/or 2, separately or together, that are suitable for oral administration may be in the form of discrete units such as hard or soft capsules, tablets, troches or lozenges, each containing a predetermined amount of the active ingredients; in the form of a dispersible powder or granules; in the form of a solution or a suspension in an aqueous liquid or non-aqueous liquid; in the form of syrups or elixirs; or in the form of an oil-in-water emulsion or a water-in-oil emulsion.

Dosage forms intended for oral use may be prepared according to any method known to the art for the manufacture of pharmaceutical formulations and such compositions.

The excipients used may be for example, (a) inert diluents such as mannitol, sorbitol, calcium carbonate, pregelatinized starch, lactose, calcium phosphate or sodium phosphate; (b) granulating and disintegrating agents, such as povidone, copovidone, hydroxypropylmethylcellulose, corn starch, alginic acid, crospovidone, sodiumstarchglycolate, croscarmellose, or polacrilin potassium; (c) binding agents such as microcrystalline cellulose or acacia; and (d) lubricating agents such as magnesium stearate, stearic acid, fumaric acid or talc.

In some cases, formulations for oral use may be in the form of hardgelatin or HPMC capsules wherein the active ingredients 1 and/or 2, separately or together, are mixed with an inert solid diluent, for example pregelatinized starch, calcium carbonate, calcium phosphate or kaolin, or dispensed via a pellet formulation. They may also be in the form of soft gelatin capsules wherein the active ingredient is mixed with water or an oil medium, for example peanut oil, liquid paraffin, medium chain triglycerides or olive oil.

The tablets, capsules or pellets may be uncoated or they may be coated by known techniques to delay disintegration and absorption in the gastrointestinal tract and thereby provide a delayed action or sustained action over a longer period. For example, a time delay material such as celluloseacetate phtalate or hydroxypropylcellulose acetate succinate or sustained release material such as ethylcellulose or ammoniomethacrylate copolymer (type B) may be employed.

Coated tablets may be prepared accordingly by coating cores produced analogously to the tablets with substances normally used for tablet coatings, for example collidone or shellac, gum arabic, talc, titanium dioxide or sugar. To achieve delayed release or prevent incompatibilities the core may also consist of a number of layers. Similarly the tablet coating may consist of a number or layers to achieve delayed release, possibly using the excipients mentioned above for the tablets.

Liquid dosage forms for oral administration include pharmaceutically acceptable emulsions, solutions, suspensions, syrups, and elixirs containing inert diluents commonly used in the art, such as water. Besides such inert diluents, compositions can also include adjuvants, such as wetting agents, emulsifying and suspending agents, and sweetening, flavoring, perfuming and preserving agents.

Aqueous suspensions normally contain the active materials 1 and/or 2, separately or together, in admixture with excipients suitable for the manufacture of aqueous suspensions. Such excipients may be (a) suspending agents such as hydroxy ethylcellulose, sodium carboxymethylcellulose, methylcellulose, hydroxypropylmethylcellulose, sodium alginate, polyvinylpyrrolidone, gum tragacanth and gum acacia; (b) dispersing or wetting agents which may be (b.1) a naturally-occurring phosphatide such as lecithin, (b.2) a condensation product of an alkylene oxide with a fatty acid, for example, polyoxyethylene stearate, (b.3) a condensation product of ethylene oxide with a long chain aliphatic alcohol, for example heptadecaethyleneoxycetanol, (b.4) a condensation product of ethylene oxide with a partial ester derived from a fatty acid and a hexitol such as polyoxyethylene sorbitol monooleate, or (b.5) a condensation product of ethylene oxide with a partial ester derived from a fatty acid and a hexitol anhydride, for example polyoxyethylene sorbitan monooleate.

The aqueous suspensions may also contain one or more preservatives, for example, ethyl or n-propyl p-hydroxybenzoate; one or more coloring agents; one or more flavoring agents; and one or more sweetening agents, such as sucrose or saccharin.

Oily suspensions may be formulated by suspending the active ingredients 1 and/or 2, separately or together, in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents and flavoring agents may be added to provide a palatable oral preparation. These compositions may be prepared by the addition of an antioxidant such as ascorbic acid.

Dispersible powders and granules are suitable for the preparation of an aqueous suspension. They provide the active ingredient 1 and/or 2, separately or together in admixture with a dispersing or wetting agent, a suspending agent and one or more preservatives. Suitable dispersing or wetting agents and suspending agents are exemplified by those already mentioned above. Additional excipients, for example, those sweetening, flavoring and coloring agents described above may also be present.

The pharmaceutical formulations, compositions, dosage forms or kit of parts of the invention may also be in the form of oil-in-water emulsions. The oily phase may be a vegetable oil such as olive oil or arachis oils, or a mineral oil such as liquid paraffin or a mixture thereof.

Suitable emulsifying agents may be (a) naturally-occurring gums such as gum acacia and gum tragacanth, (b) naturally-occurring phosphatides such as soybean and lecithin, (c) esters or partial esters derived from fatty acids and hexitol anhydrides, for example, sorbitan monooleate, (d) condensation products of said partial esters with ethylene oxide, for example polyoxyethylene sorbitan monooleate. The emulsions may also contain sweetening and flavoring agents.

Syrups and elixirs may be formulated with sweetening agents, for example, glycerol, propylene glycol, sorbitol or sucrose. Such formulations may also contain a preservative and flavoring and coloring agents.

The pharmaceutical formulations, compositions, dosage forms or kit of parts containing 1 and/or 2, separately or together may be in the form of a sterile injectable aqueous or oleagenous suspension or solution. The suspension may be formulated according to known methods using those suitable dispersing or wetting agents and suspending agents which have been mentioned above. The sterile injectable preparation may also be a sterile injectable solution or suspension in a non toxic parenterally-acceptable diluent or solvent, for example as a solution in 1,3-butane-diol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

Preparations according to this invention containing 1 and/or 2, separately or together, for parenteral administration include sterile aqueous or non-aqueous solutions, suspension, or emulsions.

Examples of non-aqueous solvents or vehicles are propylene glycol, polyethylene glycol, vegetable oils, such as olive oil and corn oil, gelatin, and injectable organic esters such as ethyl oleate. Such dosage forms may also contain adjuvants such as preserving, wetting, emulsifying, and dispersing agents. They may be sterilized by, for example, filtration through a bacteria-retaining filter, by incorporating sterilizing agents into the compositions, by irradiating the compositions, or by heating the compositions. They can also be manufactured in the form of sterile solid compositions which can be reconstituted in sterile water, or some other sterile injectable medium immediately before use. The combination of this invention may also be administered in the form of suppositories for rectal administration. This composition can be prepared by mixing the drugs with a suitable non-irritating excipient which is solid at ordinary temperatures but liquid at the rectal temperature and will therefore melt in the rectum to release the drug. Such materials are cocoa butter, hard fat, and polyethylene glycols. Compositions for buccal, nasal or sublingual administration are also prepared with standard excipients well known in the art.

For topical administration the formulations, compositions, dosage forms or kit of parts of this invention containing 1 and/or 2, separately or together may be formulated in liquid or semi-liquid preparations such as liniments, lotions, applications; oil-in-water or water-in-oil emulsions such as creams, ointments, jellies or pastes, including tooth-pastes; or solutions or suspensions such as drops, and the like.

The dosage of the active ingredients in the compositions of this invention may be varied. However, it is necessary that the amount of the active ingredient 1 for the administration as a monotherapy or the active ingredients 1 and 2, for the administration as a combination therapy, be such that a suitable dosage form is obtained. The selected dosage and the dosage form depend upon the desired therapeutic effect, on the route of administration and on the duration of the treatment. Dosage ranges in the combination are approximately one tenth to one times the clinically effective ranges required to induce the desired therapeutic effect, respectively when the compounds are used singly.

The beneficial effects of the compounds of formula (I) 1, optionally in form of the free base or in form of the pharmacologically acceptable acid addition salts thereof, can be observed regardless of whether the disturbance existed lifelong or was acquired, and independent of etiologic origin (organic—both, physically and drug induced-, psychogen, a combination of organic—both, physically and drug induced-, and psychogen, or unknown).

Within the instant invention the compounds of formula (I) 1 are preferably administered in such an amount that per single dosage between 0.01 to 400 mg of invention the compounds of formula (I) 1 are applied. Preferred are ranges of between 0.1 to 300 mg, more preferred between 0.1 to 200 mg and particularly preferred 0.1 to 50 mg of the compounds of formula (I) 1. Suitable dosage forms may contain for instance 0.01, 0.05, 0.1, 0.5, 1, 5, 10, 15, 20, 25, 30, 35, 40, 45, 50, 55, 60, 65, 70, 75, 80, 85, 90, 95, 100, 200, 300 or 400 mg of the compounds of formula (I) 1. The aforementioned values are based on the compounds of formula (I) 1 in form of the free base. If the compounds of formula (I) 1 are applied in form of one of its acid addition salts, the corresponding values are readily calculable from the aforementioned values.

Within the instant invention the antimuscarinic agents 2a are preferably administered in such an amount that per day between 0.01 to 200 mg are applied. Preferred are ranges of between 0.5 to 100 mg, particular preferred 1 to 50 mg of the antimuscarinic agents 2a. Suitable dosage forms may contain for instance 0.01, 0.05, 0.5, 1, 2, 5, 10, 20, 25, 50, 100 or 200 mg of the antimuscarinic agents 2a. Advantageously, the compounds 2a of the present invention may be administered in a single daily dose, or the total daily dosage may be administered in divided doses of two, three or four times daily.

Within the instant invention the vasopressin agonist 2b are preferably administered in such an amount that per day between 0.01 to 100 mg are applied. Preferred are ranges of between 0.5 to 100 mg, particular preferred 1 to 50 mg of the vasopressin agonist 2b. Suitable dosage forms may contain for instance 0.01, 0.05, 0.5, 1, 2, 5, 10, 20, 25, 50 or 100 mg of the vasopressin agonist 2b. Advantageously, the compounds 2b of the present invention may be administered in a single daily dose, or the total daily dosage may be administered in divided doses of two, three or four times daily.

Within the instant invention the Serotonin/Noradrenaline modulators 2c are preferably administered in such an amount that per day between 0.1 to 200 mg are applied. Preferred are ranges of between 0.5 to 150 mg, particular preferred 1 to 100 mg of the Serotonin/Noradrenaline modulators 2c. Suitable dosage forms may contain for instance 0.1, 0.5, 1, 2, 5, 10, 20, 25, 50, 100 or 200 mg of the Serotonin/Noradrenaline modulators 2c. Advantageously, the compounds 2c of the present invention may be administered in a single daily dose, or the total daily dosage may be administered in divided doses of two, three or four times daily.

In another embodiment the invention relates to a method for the treatment or prevention of urinary incontinence, comprising the administration of a therapeutically effective amount of one or more, preferably one compound of formula (I) 1, optionally in form of the free base or in form of the pharmacologically acceptable acid addition salts thereof, in combination with a therapeutically effective amount of one or more, preferably one active ingredient 2, optionally in form of the pharmaceutically acceptable salts, in form of the hydrates and/or solvates and optionally in the form of the individual optical isomers, mixtures of the individual enantiomers or racemates thereof, separately or together within one pharmaceutical composition. Preferably 1 is selected from the group consisting of the compounds (I.a), (I.b), (I.c), (I.d), (I.e), (I.f), (I.g) and (I.h).

In another embodiment the invention relates to a method for the treatment or prevention of overactive bladder syndrome, comprising the administration of a therapeutically effective amount of one or more, preferably one compound of formula (I) 1, optionally in form of the free base or in form of the pharmacologically acceptable acid addition salts thereof, in combination with a therapeutically effective amount of one or more, preferably one active ingredient 2, optionally in form of the pharmaceutically acceptable salts, in form of the hydrates and/or solvates and optionally in the form of the individual optical isomers, mixtures of the individual enantiomers or racemates thereof, separately or together within one pharmaceutical composition. Preferably 1 is selected from the group consisting of the compounds (I.a), (I.b), (I.c), (I.d), (I.e), (I.f), (I.g) and (I.h).

In another embodiment the invention relates to a method for the treatment or prevention of urge incontinence, comprising the administration of a therapeutically effective amount of one or more, preferably one compound of formula (I) 1, optionally in form of the free base or in form of the pharmacologically acceptable acid addition salts thereof, in combination with a therapeutically effective amount of one or more, preferably one active ingredient 2, optionally in form of the pharmaceutically acceptable salts, in form of the hydrates and/or solvates and optionally in the form of the individual optical isomers, mixtures of the individual enantiomers or racemates thereof, separately or together within one pharmaceutical composition. Preferably 1 is selected from the group consisting of the compounds (I.a), (I.b), (I.c), (I.d), (I.e), (I.f), (I.g) and (I.h).

In another embodiment the invention relates to a method for the treatment or prevention of stress incontinence, comprising the administration of a therapeutically effective amount of one or more, preferably one compound of formula (I) 1, optionally in form of the free base or in form of the pharmacologically acceptable acid addition salts thereof, in combination with a therapeutically effective amount of one or more, preferably one active ingredient 2, optionally in form of the pharmaceutically acceptable salts, in form of the hydrates and/or solvates and optionally in the form of the individual optical isomers, mixtures of the individual enantiomers or racemates thereof, separately or together within one pharmaceutical composition. Preferably 1 is selected from the group consisting of the compounds (I.a), (I.b), (I.c), (I.d), (I.e), (I.f), (I.g) and (I.h).

In another embodiment the invention relates to a method for the treatment or prevention of mixed incontinence, comprising the administration of a therapeutically effective amount of one or more, preferably one compound of formula (I) 1, optionally in form of the free base or in form of the pharmacologically acceptable acid addition salts thereof, in combination with a therapeutically effective amount of one or more, preferably one active ingredient 2, optionally in form of the pharmaceutically acceptable salts, in form of the hydrates and/or solvates and optionally in the form of the individual optical isomers, mixtures of the individual enantiomers or racemates thereof, separately or together within one pharmaceutical composition. Preferably 1 is selected from the group consisting of the compounds (I.a), (I.b), (I.c), (I.d), (I.e), (I.f), (I.g) and (I.h).

Another embodiment of the present invention relates to the use of the combinations of one or more, preferably one compound of formula (I) 1, optionally in form of the free base or in form of the pharmacologically acceptable acid addition salts thereof, and of one or more, preferably one active ingredient 2, optionally in form of the pharmaceutically acceptable salts, in form of the hydrates and/or solvates and optionally in the form of the individual optical isomers, mixtures of the individual enantiomers or racemates thereof, for the preparation of a medicament for the treatment of any of the aforementioned disorders. Preferably 1 is selected from the group consisting of the compounds (I.a), (I.b), (I.c), (I.d), (I.e), (I.f), (I.g) and (I.h).

Another embodiment of the present invention relates to the use of one or more, preferably one compound of formula (I) 1, optionally in form of the free base or in form of the pharmacologically acceptable acid addition salts thereof, in the manufacture of a medicament for the treatment of any of the aforementioned disorders in combination with one or more, preferably one active ingredient 2, optionally in form of the pharmaceutically acceptable salts, in form of the hydrates and/or solvates and optionally in the form of the individual optical isomers, mixtures of the individual enantiomers or racemates thereof. Preferably 1 is selected from the group consisting of the compounds (I.a), (I.b), (I.c), (I.d), (I.e), (I.f), (I.g) and (I.h).

In another embodiment the invention relates to a method for the treatment or prevention of one of the aforementioned diseases selected from the group consisting of urinary incontinence, overactive bladder syndrome, urge incontinence, stress incontinence and mixed incontinence, comprising the administration of a therapeutically effective amount of one or more, preferably one compound of formula (I) 1, optionally in form of the free base or in form of the pharmacologically acceptable acid addition salts thereof, in combination with a therapeutically effective amount of one or more, preferably one antimuscarinic agents 2a, optionally in form of the pharmaceutically acceptable salts, in form of the hydrates and/or solvates and optionally in the form of the individual optical isomers, mixtures of the individual enantiomers or racemates thereof, separately or together within one pharmaceutical composition. Preferred antimuscarinic agents 2a include Tolterodine, Oxybutynin, Solifenacin and Trospium. Preferably 1 is selected from the group consisting of the compounds (I.a), (I.b), (I.c), (I.d), (I.e), (I.f), (I.g) and (I.h).

Another embodiment of the present invention relates to the use of the combinations of one or more, preferably one compound of formula (I) 1, optionally in form of the free base or in form of the pharmacologically acceptable acid addition salts thereof, and of one or more, preferably one antimuscarinic agents 2a, optionally in form of the pharmaceutically acceptable salts, in form of the hydrates and/or solvates and optionally in the form of the individual optical isomers, mixtures of the individual enantiomers or racemates thereof, for the preparation of a medicament for the treatment of any of the aforementioned disorders. Preferred antimuscarinic agents 2a include Tolterodine, Oxybutynin, Solifenacin and Trospium. Preferably 1 is selected from the group consisting of the compounds (I.a), (I.b), (I.c), (I.d), (I.e), (I.f), (I.g) and (I.h).

Another embodiment of the present invention relates to the use of one or more, preferably one compound of formula (I) 1, optionally in form of the free base or in form of the pharmacologically acceptable acid addition salts thereof, in the manufacture of a medicament for the treatment of any of the aforementioned disorders in combination with one or more, preferably one antimuscarinic agent 2a, optionally in form of the pharmaceutically acceptable salts, in form of the hydrates and/or solvates and optionally in the form of the individual optical isomers, mixtures of the individual enantiomers or racemates thereof. Preferred antimuscarinic agents 2a include Tolterodine, Oxybutynin, Solifenacin and Trospium. Preferably 1 is selected from the group consisting of the compounds (I.a), (I.b), (I.c), (I.d), (I.e), (I.f), (I.g) and (I.h).

In another embodiment the invention relates to a method for the treatment or prevention of one of the aforementioned diseases selected from the group consisting of urinary incontinence, overactive bladder syndrome, urge incontinence, stress incontinence and mixed incontinence, comprising the administration of a therapeutically effective amount of one or more, preferably one compound of formula (I) 1, optionally in form of the free base or in form of the pharmacologically acceptable acid addition salts thereof, in combination with a therapeutically effective amount of one or more, preferably one vasopressin agonist 2b, optionally in form of the pharmaceutically acceptable salts, in form of the hydrates and/or solvates and optionally in the form of the individual optical isomers, mixtures of the individual enantiomers or racemates thereof, separately or together within one pharmaceutical composition. A preferred vasopressin agonist 2b is Desmopressin. Preferably 1 is selected from the group consisting of the compounds (I.a), (I.b), (I.c), (I.d), (I.e), (I.f), (I.g) and (I.h).

Another embodiment of the present invention relates to the use of the combinations of one or more, preferably one compound of formula (I) 1, optionally in form of the free base or in form of the pharmacologically acceptable acid addition salts thereof, and of one or more, preferably one vasopressin agonist 2b, optionally in form of the pharmaceutically acceptable salts, in form of the hydrates and/or solvates and optionally in the form of the individual optical isomers, mixtures of the individual enantiomers or racemates thereof, for the preparation of a medicament for the treatment of any of the aforementioned disorders. A preferred vasopressin agonist 2b is Desmopressin. Preferably 1 is selected from the group consisting of the compounds (I.a), (I.b), (I.c), (I.d), (I.e), (I.f), (I.g) and (I.h).

Another embodiment of the present invention relates to the use of one or more, preferably one compound of formula (I) 1, optionally in form of the free base or in form of the pharmacologically acceptable acid addition salts thereof, in the manufacture of a medicament for the treatment of any of the aforementioned disorders in combination with one or more, preferably one vasopressin agonist 2b, optionally in form of the pharmaceutically acceptable salts, in form of the hydrates and/or solvates and optionally in the form of the individual optical isomers, mixtures of the individual enantiomers or racemates thereof. A preferred vasopressin agonist 2b is Desmopressin. Preferably 1 is selected from the group consisting of the compounds (I.a), (I.b), (I.c), (I.d), (I.e), (I.f), (I.g) and (I.h).

In another embodiment the invention relates to a method for the treatment or prevention of one of the aforementioned diseases selected from the group consisting of urinary incontinence, overactive bladder syndrome, urge incontinence, stress incontinence and mixed incontinence, comprising the administration of a therapeutically effective amount of one or more, preferably one compound of formula (I) 1, optionally in form of the free base or in form of the pharmacologically acceptable acid addition salts thereof, in combination with a therapeutically effective amount of one or more, preferably one Serotonin/Noradrenaline modulator 2c, optionally in form of the pharmaceutically acceptable salts, in form of the hydrates and/or solvates and optionally in the form of the individual optical isomers, mixtures of the individual enantiomers or racemates thereof, separately or together within one pharmaceutical composition. Preferred Serotonin/Noradrenaline modulators 2c include Venlafaxine, Duloxetine, Reboxetine and Cizoliritine. Preferably 1 is selected from the group consisting of the compounds (I.a), (I.b), (I.c), (I.d), (I.e), (I.f), (I.g) and (I.h).

Another embodiment of the present invention relates to the use of the combinations of one or more, preferably one compound of formula (I) 1, optionally in form of the free base or in form of the pharmacologically acceptable acid addition salts thereof, and of one or more, preferably one Serotonin/Noradrenaline modulator 2c, optionally in form of the pharmaceutically acceptable salts, in form of the hydrates and/or solvates and optionally in the form of the individual optical isomers, mixtures of the individual enantiomers or racemates thereof, for the preparation of a medicament for the treatment of any of the aforementioned disorders. Preferred Serotonin/Noradrenaline modulators 2c include Venlafaxine, Duloxetine, Reboxetine and Cizoliritine. Preferably 1 is selected from the group consisting of the compounds (I.a), (I.b), (I.c), (I.d), (I.e), (I.f), (I.g) and (I.h).

Another embodiment of the present invention relates to the use of one or more, preferably one compound of formula (I) 1, optionally in form of the free base or in form of the pharmacologically acceptable acid addition salts thereof, in the manufacture of a medicament for the treatment of any of the aforementioned disorders in combination with one or more, preferably one Serotonin/Noradrenaline modulator 2c, optionally in form of the pharmaceutically acceptable salts, in form of the hydrates and/or solvates and optionally in the form of the individual optical isomers, mixtures of the individual enantiomers or racemates thereof. Preferred Serotonin/Nor-adrenaline modulators 2c include Venlafaxine, Duloxetine, Reboxetine and Cizoliritine. Preferably 1 is selected from the group consisting of the compounds (I.a), (I.b), (I.c), (I.d), (I.e), (I.f), (I.g) and (I.h).

In a preferred embodiment the invention relates to a method for the treatment or prevention of overactive bladder syndrome, comprising the administration of a therapeutically effective amount of one or more, preferably one compound of formula (I) 1, optionally in form of the free base or in form of the pharmacologically acceptable acid addition salts thereof, in combination with a therapeutically effective amount of one or more, preferably one antimuscarinic agents 2a, selected from the group consisting of Tolterodine, Oxybutynin, Solifenacin and Trospium, optionally in form of the pharmaceutically acceptable salts, in form of the hydrates and/or solvates and optionally in the form of the individual optical isomers, mixtures of the individual enantiomers or racemates thereof, separately or together within one pharmaceutical composition. Preferably 1 is selected from the group consisting of the compounds (I.a), (I.b), (I.c), (I.d), (I.e), (I.f), (I.g) and (I.h).

Another embodiment of the present invention relates to the use of the combinations of one or more, preferably one compound of formula (I) 1, optionally in form of the free base or in form of the pharmacologically acceptable acid addition salts thereof, and of one or more, preferably one antimuscarinic agent 2a, selected from the group consisting of Tolterodine, Oxybutynin, Solifenacin and Trospium, optionally in form of the pharmaceutically acceptable salts, in form of the hydrates and/or solvates and optionally in the form of the individual optical isomers, mixtures of the individual enantiomers or racemates thereof, for the preparation of a medicament for the treatment of the overactive bladder syndrome. Preferably 1 is selected from the group consisting of the compounds (I.a), (I.b), (I.c), (I.d), (I.e), (I.f), (I.g) and (I.h).

Another embodiment of the present invention relates to the use of one or more, preferably one compound of formula (I) 1, optionally in form of the free base or in form of the pharmacologically acceptable acid addition salts thereof, in the manufacture of a medicament for the treatment of overactive bladder syndrome in combination with one or more, preferably one antimuscarinic agent 2a, selected from the group consisting of Tolterodine, Oxybutynin, Solifenacin and Trospium, optionally in form of the pharmaceutically acceptable salts, in form of the hydrates and/or solvates and optionally in the form of the individual optical isomers, mixtures of the individual enantiomers or racemates thereof. Preferably 1 is selected from the group consisting of the compounds (I.a), (I.b), (I.c), (I.d), (I.e), (I.f), (I.g) and (I.h).

In a preferred embodiment the invention relates to a method for the treatment or prevention of urge incontinence, comprising the administration of a therapeutically effective amount of one or more, preferably one compound of formula (I) 1, optionally in form of the free base or in form of the pharmacologically acceptable acid addition salts thereof, in combination with a therapeutically effective amount of one or more, preferably one antimuscarinic agents 2a, selected from the group consisting of Tolterodine, Oxybutynin, Solifenacin and Trospium, optionally in form of the pharmaceutically acceptable salts, in form of the hydrates and/or solvates and optionally in the form of the individual optical isomers, mixtures of the individual enantiomers or racemates thereof, separately or together within one pharmaceutical composition. Preferably 1 is selected from the group consisting of the compounds (I.a), (I.b), (I.c), (I.d), (I.e), (I.f), (I.g) and (I.h).

Another embodiment of the present invention relates to the use of the combinations of one or more, preferably one compound of formula (I) 1, optionally in form of the free base or in form of the pharmacologically acceptable acid addition salts thereof, and of one or more, preferably one antimuscarinic agent 2a, selected from the group consisting of Tolterodine, Oxybutynin, Solifenacin and Trospium, optionally in form of the pharmaceutically acceptable salts, in form of the hydrates and/or solvates and optionally in the form of the individual optical isomers, mixtures of the individual enantiomers or racemates thereof, for the preparation of a medicament for the treatment of urge incontinence. Preferably 1 is selected from the group consisting of the compounds (I.a), (I.b), (I.c), (I.d), (I.e), (I.f), (I.g) and (I.h).

Another embodiment of the present invention relates to the use of one or more, preferably one compound of formula (I) 1, optionally in form of the free base or in form of the pharmacologically acceptable acid addition salts thereof, in the manufacture of a medicament for the treatment of urge incontinence in combination with one or more, preferably one antimuscarinic agent 2a, selected from the group consisting of Tolterodine, Oxybutynin, Solifenacin and Trospium, optionally in form of the pharmaceutically acceptable salts, in form of the hydrates and/or solvates and optionally in the form of the individual optical isomers, mixtures of the individual enantiomers or racemates thereof. Preferably 1 is selected from the group consisting of the compounds (I.a), (I.b), (I.c), (I.d), (I.e), (I.f), (I.g) and (I.h).

In a preferred embodiment the invention relates to a method for the treatment or prevention of overactive bladder syndrome, comprising the administration of a therapeutically effective amount of one or more, preferably one compound of formula (I) 1, optionally in form of the free base or in form of the pharmacologically acceptable acid addition salts thereof, in combination with a therapeutically effective amount of the vasopressin agonist 2b Desmopressin, optionally in form of the pharmaceutically acceptable salts, in form of the hydrates and/or solvates and optionally in the form of the individual optical isomers, mixtures of the individual enantiomers or racemates thereof, separately or together within one pharmaceutical composition. Preferably 1 is selected from the group consisting of the compounds (I.a), (I.b), (I.c), (I.d), (I.e), (I.f), (I.g) and (I.h).

Another embodiment of the present invention relates to the use of the combinations of one or more, preferably one compound of formula (I) 1, optionally in form of the free base or in form of the pharmacologically acceptable acid addition salts thereof, and of the vasopressin agonist 2b Desmopressin, optionally in form of the pharmaceutically acceptable salts, in form of the hydrates and/or solvates and optionally in the form of the individual optical isomers, mixtures of the individual enantiomers or racemates thereof, for the preparation of a medicament for the treatment of the overactive bladder syndrome. Preferably 1 is selected from the group consisting of the compounds (I.a), (I.b), (I.c), (I.d), (I.e), (I.f), (I.g) and (I.h).

Another embodiment of the present invention relates to the use of one or more, preferably one compound of formula (I) 1, optionally in form of the free base or in form of the pharmacologically acceptable acid addition salts thereof, in the manufacture of a medicament for the treatment of the overactive bladder syndrome in combination with the vasopressin agonist 2b Desmopressin, optionally in form of the pharmaceutically acceptable salts, in form of the hydrates and/or solvates and optionally in the form of the individual optical isomers, mixtures of the individual enantiomers or racemates thereof. Preferably 1 is selected from the group consisting of the compounds (I.a), (I.b), (I.c), (I.d), (I.e), (I.f), (I.g) and (I.h).

In a preferred embodiment the invention relates to a method for the treatment or prevention of urge incontinence, comprising the administration of a therapeutically effective amount of one or more, preferably one compound of formula (I) 1, optionally in form of the free base or in form of the pharmacologically acceptable acid addition salts thereof, in combination with a therapeutically effective amount of the vasopressin agonist 2b Desmopressin, optionally in form of the pharmaceutically acceptable salts, in form of the hydrates and/or solvates and optionally in the form of the individual optical isomers, mixtures of the individual enantiomers or racemates thereof, separately or together within one pharmaceutical composition. Preferably 1 is selected from the group consisting of the compounds (I.a), (I.b), (I.c), (I.d), (I.e), (I.f), (I.g) and (I.h).

Another embodiment of the present invention relates to the use of the combinations of one or more, preferably one compound of formula (I) 1, optionally in form of the free base or in form of the pharmacologically acceptable acid addition salts thereof, and of the vasopressin agonist 2b Desmopressin, optionally in form of the pharmaceutically acceptable salts, in form of the hydrates and/or solvates and optionally in the form of the individual optical isomers, mixtures of the individual enantiomers or racemates thereof, for the preparation of a medicament for the treatment of urge incontinence. Preferably 1 is selected from the group consisting of the compounds (I.a), (I.b), (I.c), (I.d), (I.e), (I.f), (I.g) and (I.h).

Another embodiment of the present invention relates to the use of one or more, preferably one compound of formula (I) 1, optionally in form of the free base or in form of the pharmacologically acceptable acid addition salts thereof, in the manufacture of a medicament for the treatment of urge incontinence in combination with the vasopressin agonist 2b Desmopressin, optionally in form of the pharmaceutically acceptable salts, in form of the hydrates and/or solvates and optionally in the form of the individual optical isomers, mixtures of the individual enantiomers or racemates thereof. Preferably 1 is selected from the group consisting of the compounds (I.a), (I.b), (I.c), (I.d), (I.e), (I.f), (I.g) and (I.h).

In a preferred embodiment the invention relates to a method for the treatment or prevention of stress incontinence, comprising the administration of a therapeutically effective amount of one or more, preferably one compound of formula (I) 1, optionally in form of the free base or in form of the pharmacologically acceptable acid addition salts thereof, in combination with a therapeutically effective amount of one or more, preferably one Serotonin/Noradrenaline modulator 2c, selected from the group consisting of Venlafaxine, Duloxetine, Reboxetine and Cizoliritine, optionally in form of the pharmaceutically acceptable salts, in form of the hydrates and/or solvates and optionally in the form of the individual optical isomers, mixtures of the individual enantiomers or racemates thereof, separately or together within one pharmaceutical composition. Preferably 1 is selected from the group consisting of the compounds (I.a), (I.b), (I.c), (I.d), (I.e), (I.f), (I.g) and (I.h).

Another embodiment of the present invention relates to the use of the combinations of one or more, preferably one compound of formula (I) 1, optionally in form of the free base or in form of the pharmacologically acceptable acid addition salts thereof, and of one or more, preferably one Serotonin/Noradrenaline modulator 2c, selected from the group consisting of Venlafaxine, Duloxetine, Reboxetine and Cizoliritine, optionally in form of the pharmaceutically acceptable salts, in form of the hydrates and/or solvates and optionally in the form of the individual optical isomers, mixtures of the individual enantiomers or racemates thereof, for the preparation of a medicament for the treatment of stress incontinence. Preferably 1 is selected from the group consisting of the compounds (I.a), (I.b), (I.c), (I.d), (I.e), (I.f), (I.g) and (I.h).

Another embodiment of the present invention relates to the use of one or more, preferably one compound of formula (I) 1, optionally in form of the free base or in form of the pharmacologically acceptable acid addition salts thereof, in the manufacture of a medicament for the treatment of stress incontinence in combination with of one or more, preferably one Serotonin/Noradrenaline modulator 2c, selected from the group consisting of Venlafaxine, Duloxetine, Reboxetine and Cizoliritine, optionally in form of the pharmaceutically acceptable salts, in form of the hydrates and/or solvates and optionally in the form of the individual optical isomers, mixtures of the individual enantiomers or racemates thereof. Preferably 1 is selected from the group consisting of the compounds (I.a), (I.b), (I.c), (I.d), (I.e), (I.f), (I.g) and (I.h).

The following examples 1) to 72) illustrate combinations of the present invention without restricting its scope:

| Example | Compound of | Compound | Example | Compound of | Compound |
|---|---|---|---|---|---|
| No. | formula (I) 1 | 2 | No. | formula (I) 1 | 2 |
| 1) | (I.a) | Tolterodine | 2) | (I.a) | Oxybutynin |
| 3) | (I.a) | Solifenacin | 4) | (I.a) | Trospium |
| 5) | (I.a) | Desmopressin | 6) | (I.a) | Venlafaxine |
| 7) | (I.a) | Duloxetine | 8) | (I.a) | Reboxetine |
| 9) | (I.a) | Cizoliritine | 10) | (I.b) | Tolterodine |
| 11) | (I.b) | Oxybutynin | 12) | (I.b) | Solifenacin |
| 13) | (I.b) | Trospium | 14) | (I.b) | Desmopressin |
| 15) | (I.b) | Venlafaxine | 16) | (I.b) | Duloxetine |
| 17) | (I.b) | Reboxetine | 18) | (I.b) | Cizoliritine |
| 19) | (I.c) | Tolterodine | 20) | (I.c) | Oxybutynin |
| 21) | (I.c) | Solifenacin | 22) | (I.c) | Trospium |
| 23) | (I.c) | Desmopressin | 24) | (I.c) | Venlafaxine |
| 25) | (I.c) | Duloxetine | 26) | (I.c) | Reboxetine |
| 27) | (I.c) | Cizoliritine | 28) | (I.d) | Tolterodine |
| 29) | (I.d) | Oxybutynin | 30) | (I.d) | Solifenacin |
| 31) | (I.d) | Trospium | 32) | (I.d) | Desmopressin |
| 33) | (I.d) | Venlafaxine | 34) | (I.d) | Duloxetine |
| 35) | (I.d) | Reboxetine | 36) | (I.d) | Cizoliritine |
| 37) | (I.e) | Tolterodine | 38) | (I.e) | Oxybutynin |
| 39) | (I.e) | Solifenacin | 40) | (I.e) | Trospium |
| 41) | (I.e) | Desmopressin | 42) | (I.e) | Venlafaxine |
| 43) | (I.e) | Duloxetine | 44) | (I.e) | Reboxetine |
| 45) | (I.e) | Cizoliritine | 46) | (I.f) | Tolterodine |
| 47) | (I.f) | Oxybutynin | 48) | (I.f) | Solifenacin |
| 49) | (I.f) | Trospium | 50) | (I.f) | Desmopressin |
| 51) | (I.f) | Venlafaxine | 52) | (I.f) | Duloxetine |
| 53) | (I.f) | Reboxetine | 54) | (I.f) | Cizoliritine |
| 55) | (I.g) | Tolterodine | 56) | (I.g) | Oxybutynin |
| 57) | (I.g) | Solifenacin | 58) | (I.g) | Trospium |
| 59) | (I.g) | Desmopressin | 60) | (I.g) | Venlafaxine |
| 61) | (I.g) | Duloxetine | 62) | (I.g) | Reboxetine |
| 63) | (I.g) | Cizoliritine | 64) | (I.h) | Tolterodine |
| 65) | (I.h) | Oxybutynin | 66) | (I.h) | Solifenacin |
| 67) | (I.h) | Trospium | 68) | (I.h) | Desmopressin |
| 69) | (I.h) | Venlafaxine | 70) | (I.h) | Duloxetine |
| 71) | (I.h) | Reboxetine | 72) | (I.h) | Cizoliritine |

Above mentioned combinations can be used for the treatment or prevention of urinary incontinence, overactive bladder syndrome, urge incontinence, stress incontinence and/or mixed incontinence.

The Examples which follow illustrate the present invention without restricting its scope:

### Examples of Pharmaceutical Formulations

| A) Tablets | per tablet | |
|---|---|---|
| compound (I.a) | 100 | mg |
| lactose | 240 | mg |
| corn starch | 340 | mg |
| polyvinylpyrrolidone | 45 | mg |
| magnesium stearate | 15 | mg |
| | 740 | mg |

The finely ground active substance, lactose and some of the corn starch are mixed together. The mixture is screened, then moistened with a solution of polyvinylpyrrolidone in water, kneaded, wet-granulated and dried. The granules, the remaining corn starch and the magnesium stearate are screened and mixed together. The mixture is compressed to produce tablets of suitable shape and size.

| B) Tablets | per tablet | |
|---|---|---|
| compound (I.b) | 80 | mg |
| corn starch | 190 | mg |
| lactose | 55 | mg |
| microcrystalline cellulose | 35 | mg |
| polyvinylpyrrolidone | 15 | mg |
| sodium-carboxymethyl starch | 23 | mg |
| magnesium stearate | 2 | mg |
| | 400 | mg |

The finely ground active substance, some of the corn starch, lactose, microcrystalline cellulose and polyvinylpyrrolidone are mixed together, the mixture is screened and worked with the remaining corn starch and water to form a granulate which is dried and screened. The sodium-carboxymethyl starch and the magnesium stearate are added and mixed in and the mixture is compressed to form tablets of a suitable size.

| C) Coated tablets | per coated tablet | |
|---|---|---|
| compound (I.c) | 5 | mg |
| corn starch | 41.5 | mg |
| lactose | 30 | mg |
| polyvinylpyrrolidone | 3 | mg |
| magnesium stearate | 0.5 | mg |
| | 80 | mg |

The active substance, corn starch, lactose and polyvinylpyrrolidone are thoroughly mixed and moistened with water. The moist mass is pushed through a screen with a 1 mm mesh size, dried at about 45° C. and the granules are then passed through the same screen. After the magnesium stearate has been mixed in, convex tablet cores with a diameter of 6 mm are compressed in a tablet-making machine. The tablet cores thus produced are coated in known manner with a covering consisting essentially of sugar and talc. The finished coated tablets are polished with wax.

| D) Capsules | per capsule | |
|---|---|---|
| compound (I.d) | 1 50 | mg |
| Corn starch | 268.5 | mg |
| Magnesium stearate | 1.5 | mg |
| | 420 | mg |

The substance and corn starch are mixed and moistened with water. The moist mass is screened and dried. The dry granules are screened and mixed with magnesium stearate. The finished mixture is packed into size 1 hard gelatine capsules.

E) Ampoule solution

| | | |
|---|---|---|
| compound (I.e) | 50 | mg |
| sodium chloride | 50 | mg |
| water for inj. | 5 | ml |

The active substance is dissolved in water at its own pH or optionally at pH 5.5 to 6.5 and sodium chloride is added to make it isotonic. The solution obtained is filtered free from pyrogens and the filtrate is transferred under aseptic conditions into ampoules which are then sterilised and sealed by fusion.

F) Suppositories

| | | |
|---|---|---|
| compound (I.f) | 50 | mg |
| solid fat | 1650 | mg |
| | 1700 | mg |

The hard fat is melted. At 40° C. the ground active substance is homogeneously dispersed. It is cooled to 38° C. and poured into slightly chilled suppository moulds.

G) Film coated tablet: Combination (I.b) with 2a

| | |
|---|---|
| Constituents | mg/tablet |

Core

| | |
|---|---|
| compound (I.b) | 50.000 |
| Tolterodine | 70.225 |
| Anhydrous dibasic calcium phosphate | 100.000 |
| Microcrystalline cellulose | 203.090 |
| HPMC (Methocel E5) | 6.615 |
| Croscarmellose sodium | 8.820 |
| Magnesium stearate | 2.250 |

Coating

| | |
|---|---|
| HPMC (Methocel E5) | 4.320 |
| Polyethylene Glycol 6000 | 1.260 |
| Titanium dioxide | 1.800 |
| Talc | 1.542 |
| Iron oxide red | 0.078 |
| Total Film coated tablet | 450.000 |

H) Film coated tablet: Combination (I.b) with 2b

| | |
|---|---|
| Constituents | mg/tablet |

Core

| | |
|---|---|
| compound (I.b) | 50.000 |
| Desmopressin | 10.000 |
| Lactose monohydrate | 133.750 |
| Microcrystalline cellulose | 40.000 |
| Hydroxypropylcellulose | 2.500 |
| Corn starch | 12.500 |
| Magnesium stearate | 1.250 |

Coating

| | |
|---|---|
| HPMC (e.g. Pharmacoat 606) | 2.400 |
| Polyethylene Glycol 6000 | 0.700 |
| Titanium dioxide | 1.000 |
| Talc | 0.857 |
| Iron oxide yellow | 0.043 |
| Total Film coated tablet | 255.000 |

I) Film coated bilayer tablet: Combination (I.c) with 2c

| | |
|---|---|
| Constituents | mg/tablet |

Core

| | |
|---|---|
| compound (I.c) | 50.000 |
| Duloxetine | 24.000 |
| Lactose monohydrate | 143.490 |
| Microcrystalline cellulose | 47.810 |
| HPMC (e.g. Pharmacoat 606) | 2.500 |
| Carboxymethylcellulose sodium | 5.000 |
| Mannitol | 60.000 |
| Corn starch | 36.500 |
| Povidone | 1.000 |
| Colloidal silicon dioxide | 1.000 |
| Magnesium stearate | 1.700 |

Coating

| | |
|---|---|
| HPMC (e.g. Methocel E5) | 3.360 |
| Polyethylene Glycol 6000 | 0.980 |
| Titanium dioxide | 1.400 |
| Talc | 1.200 |
| Iron oxide red | 0.060 |
| Total Film coated bilayer tablet | 380.000 |

1-27. (canceled)

28) A method of treating urinary incontinence, comprising the administration of a therapeutically effective amount of a compound of formula (I) 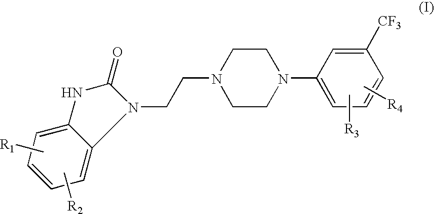
wherein R₁, R₂, R₃, and R₄ denote hydrogen or hydroxy with the proviso that R₁, R₂, R₃, and R₄ cannot simultaneously represent hydrogen,
optionally in form of the free base or a pharmacologically acceptable acid addition salt thereof, optionally in combination with a pharmaceutically acceptable excipient.

29) The method according to claim 28, wherein a compound of formula (I) 1, optionally in form of the free base or of a pharmacologically acceptable acid addition salt thereof, is administered in combination with a therapeutically effective amount of another active ingredient 2, optionally in combination with a pharmaceutically acceptable excipient.

30) The method according to claim 28, wherein the compound of formula (I) is selected from the group consisting of 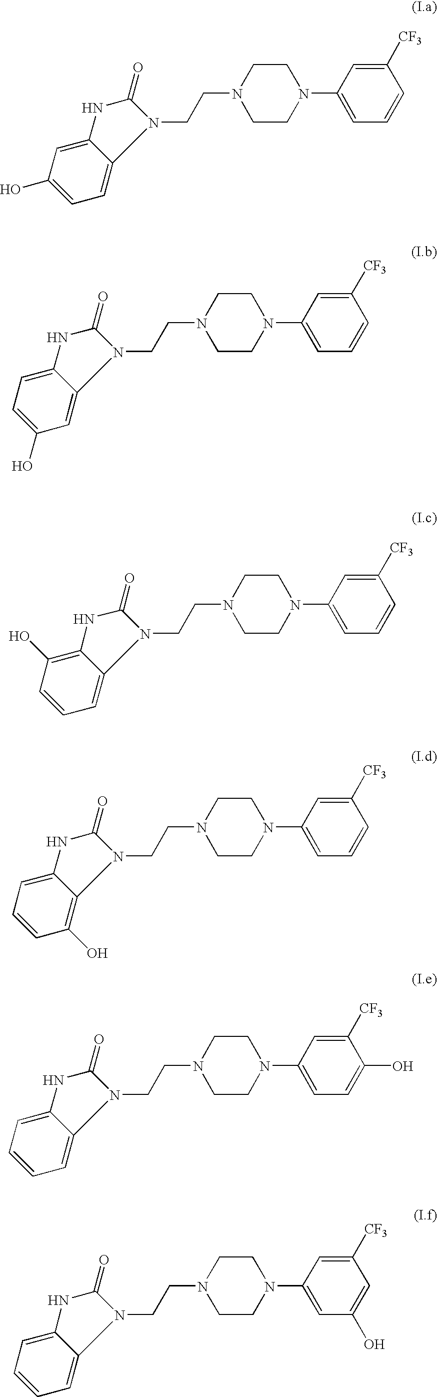 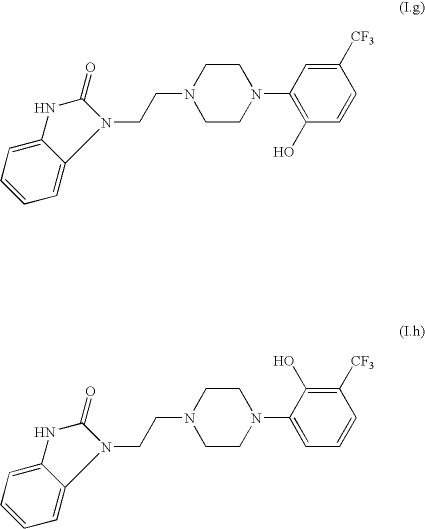
optionally in form of the tree base or a pharmacologically acceptable acid addition salt thereof.

31) The method according to claim 28, wherein the urinary incontinence is overactive bladder syndrome.

32) The method according to claim 28, wherein the urinary incontinence is urge incontinence.

33) The method according to claim 28, wherein the urinary incontinence is stress incontinence.

34) The method according to claim 28, wherein the urinary incontinence is mixed incontinence.

35) The method according to claim 29, wherein the active ingredient 2 is selected from the group consisting of antimuscarinic agents 2a, vasopressin agonists 2b and Serotonin/Noradrenaline modulators 2c.

36) The method according to claims 35, wherein the active ingredient 2 is an antimuscarinic agent 2a.

37) The method according to claim 36, wherein the antimuscarinic agent 2a is selected from the group consisting of Tolterodine, Oxybutynin, Solifenacin, Trospium, and the pharmaceutically acceptable acid addition salts thereof.

38) The method according to claims 35, wherein the active ingredient 2 is a vasopressin agonist 2b.

39) The method according to claim 38, wherein the vasopressin agonists 2b is desmopressin or a pharmaceutically acceptable acid addition salt thereof.

40) The method according to claim 35, wherein the active ingredient 2 is a Serotonin/Noradrenaline modulator 2c.

41) The method according to claim 40, wherein the Serotonin/Noradrenaline modulator 2c is selected from the group consisting of Venlafaxine, Duloxetine, Reboxetine, Cizoliritine, and the pharmaceutically acceptable acid addition salts thereof.

42) A pharmaceutical composition comprising a therapeutically effective amount of a compound of formula (I) 1 as one active ingredient, wherein R₁, R₂, R₃, and R₄ denote hydrogen or hydroxy with the proviso that R₁, R₂, R₃, and R₄ cannot simultaneously represent hydrogen, optionally in form of the free base or a pharmacologically acceptable acid addition salt thereof, in combination with a therapeutically effective amount an active ingredient L optionally in combination with a pharmaceutically acceptable excipient.

43) The pharmaceutical composition according to claim 42, wherein the compound of formula (I) 1 is selected from the group consisting of compounds (I.a), (I.b), (I.c), (I.d), (I.e), (I.f), (I.g) and (I.h), optionally in form of the free base or a pharmacologically acceptable acid addition salt thereof.

44) The pharmaceutical composition according to claim 42, wherein the active ingredient 2 is selected from the group consisting of antimuscarinic agents 2a, vasopressin agonists 2b and Serotonin/Noradrenaline modulators 2c.

45) The pharmaceutical composition according to claim 44, wherein the active ingredient 2 is an antimuscarinic agent 2a.

46) The pharmaceutical composition according to claim 45, wherein the antimuscarinic agent 2a is selected from the group consisting of Tolterodine, Oxybutynin, Solifenacin and Trospium, and the pharmaceutically acceptable acid addition salts thereof.

47) The pharmaceutical composition according to claim 44, wherein the active ingredient 2 is a vasopressin agonist 2b.

48) The pharmaceutical composition according to claim 47, wherein the vasopressin agonist 2b is desmopressin or a pharmaceutically acceptable acid addition salt thereof.

49) The pharmaceutical composition according to claims 44, wherein the active ingredient 2 is a Serotonin/Noradrenaline modulator 2c.

50) The pharmaceutical composition according to claim 49, wherein the Serotonin/Noradrenaline modulator 2c is selected from the group consisting of Venlafaxine, Duloxetine, Reboxetine and Cizoliritine, and the pharmaceutically acceptable acid addition salts thereof.

51) The pharmaceutical composition according to claims 42 wherein the active ingredients 1 and 2 are together in one dosage form.

52) The pharmaceutical composition according to claims 42 wherein the active ingredients 1 and 2 are separate, each in one dosage form.
